(19) United States
(12) Patent Application Publication
(10) Pub. No.: US 2026/0114935 A1
(43) Pub. Date: Apr. 30, 2026

(54) ULTRASOUND NEEDLE NAVIGATION SYSTEMS WITH ADAPTIVE DERIVATION FOR TWO UNCERTAIN TRANSFORMATION MATRICES

(71) Applicant: National Taiwan University Hospital, Taipei City (TW)

(72) Inventors: Cheng-Yuan HSIEH, Taipei City (TW); Yu-Shun WANG, Hsinchu County (TW); Han-Po YANG, Taoyuan City (TW)

(21) Appl. No.: 19/367,067

(22) Filed: Oct. 23, 2025

Related U.S. Application Data
(60) Provisional application No. 63/711,354, filed on Oct. 24, 2024.

(51) Int. Cl. A61B 34/20; A61B 8/08

(52) U.S. Cl. CPC A61B 34/20; A61B 8/0841; A61B 2034/2063

(57) ABSTRACT

The provided ultrasound needle navigation system includes a tracking station, a transducer tracker disposed on the tail of an ultrasound transducer, a needle tracker disposed on the tail of a needle, a needle image detection module, and a processing unit. The needle image detection module acquires a dynamic sequence of measured needle shaft coordinate system from an ultrasound image sequence. The processing unit generates multiple estimated transducer tracker to scanning plane transformation matrices and multiple estimated needle tracker to needle tip transformation matrices, thereby generating multiple sequences of estimated needle shaft coordinate systems. To minimize the estimation losses between the measured and estimated needle shaft coordinate systems, the system iteratively updates the estimated transformation matrices to obtain a final transducer tracker to scanning plane transformation matrix and a final needle tracker to needle tip transformation matrix, thereby enabling three-dimensional needle navigation across different ultrasound transducers and needles.

### CROSS-REFERENCE TO RELATED APPLICATIONS

This application claims the benefit of the filing date of U.S. Provisional Application No. 63/711,354, filed Oct. 24, 2024, under 35 U.S.C. § 119(e). The subject matter of the foregoing application is incorporated herein by reference.

### BACKGROUND

### Field of the Disclosure

The present invention relates to an ultrasound instrument navigation system and method, particularly an ultrasound needle navigation system and method used for guiding needle puncture procedures.

### Description of Related Art

In clinical procedures such as ultrasound-guided aspiration or puncture, a needle or needle-like instrument may be inserted into a patient's body. To ensure the needle follows correct orientation (direction of advancement), an ultrasound transducer can be used to detect the needle's position and orientation. To achieve this safely, the user must place the entire needle or needle-like instrument within the two-dimensional (2D) scanning plane of the ultrasound, which is challenging since the needle tip may pierce out of the ultrasound's thin 2D scanning plane. To address this difficulty, a three-dimensional (3D) tracking and navigation system, using needle and transducer trackers mounted on their respective instruments, can display the time series pose of both devices, facilitating manual adjustments.

Generally, a 3D navigation system typically comprises multiple coordinate systems, including the tracking field generator coordinate system, transducer tracker coordinate system, scanning plane coordinate system, needle tip coordinate system, and needle tracker coordinate system. It also includes multiple transformation matrices: tracking field generator to transducer tracker matrix, transducer tracker to scanning plane matrix, scanning plane to needle tip matrix, needle tracker to needle tip matrix, and needle tracker to tracking field generator matrix. The purpose of this navigation system is to map the needle tip's position in space to a target coordinate position (such as the scanning plane). These five transformation matrices exhibit closure properties, meaning that when starting from the tracking field generator position and transforming through all five matrices, one returns to the original tracking field generator position.

In existing technology, a 3D navigation system must obtain information on at least four of the transformation matrices to calculate the fifth transformation matrix for ultrasound navigation of the needle. However, when different specifications of transducers are used, the transducer tracker to scanning plane transformation matrix changes; similarly, when different specifications of needles are used, the needle tracker to needle tip transformation matrix changes. In such cases, the system must obtain the corresponding updated transformation matrices for operation, which limits users to transducers and/or needles with known transformation matrix information, thereby restricting the system's versatility.

Consequently, there is a need for an innovative ultrasound needle navigation system and method to overcome these limitations.

### SUMMARY

The present invention relates to an ultrasound needle navigation system capable of improving accuracy and efficiency in determining transformation relationships among an ultrasound transducer, a needle, and a scanning plane during puncture navigation. The system includes a transducer tracker, a needle tracker, a tracking station, a needle image detection module, and a processing unit. The transducer tracker is installed at a tail of an ultrasound transducer, and is corresponding to a transducer tracker coordinate system (CS2), wherein the ultrasound transducer is configured to provide a scanning plane, corresponding to a scanning plane coordinate system (CS3). The needle tracker is installed at a tail of a needle, and is corresponding to a needle tracker coordinate system, wherein the needle is configured to provide a tip, corresponding to a needle tip coordinate system. The tracking station is configured to generate a tracking field through a tracking field generator (e.g. a tracking field generator coordinate system (CS1)), and provide a dynamic sequence of the transducer tracker coordinate system (CS2) and a dynamic sequence of the needle tracker coordinate system (CS5) to a processing unit. The needle image detection module is configured to obtain a dynamic sequence of a measured needle shaft coordinate system (CS6) of the needle in the scanning plane from ultrasound image sequence detected by the ultrasound transducer, and obtain a scanning plane to measured needle shaft transformation matrix (M3′). The processing unit is configured to generate multiple estimated transducer tracker to scanning plane transformation matrices (M2′) and multiple estimated needle tracker to needle tip transformation matrices (M4′) based on the dynamic sequence of the transducer tracker coordinate system (CS2), the dynamic sequence of the measured needle shaft coordinate system (CS6), and the dynamic sequence of the needle tracker coordinate system (CS5). Wherein, the processing unit is further configured to generate multiple dynamic sequences of estimated needle shaft coordinate systems (CS6′) based on the multiple estimated transducer tracker to scanning plane transformation matrices (M2′) and the multiple estimated needle tracker to needle tip transformation matrices(M4′), and calculate estimation losses and perform iterative computation using the multiple dynamic sequences of the estimated needle shaft coordinate systems (CS6′) and the dynamic sequence of the measured needle shaft coordinate system (CS6) to determine a final transducer tracker to scanning plane transformation matrix (M2) and a final needle tracker to needle tip transformation matrix (M4).

Optionally, the processing unit is connected to the tracking station, and the tracking station can provide a dynamic sequence of a transducer tracker to field generator transformation matrix and a dynamic sequence of the needle tracker to the field generator transformation matrix. Optionally, the needle image detection module detects a needle shaft image in the thickness of the ultrasound transducer scanning plane (60), and generates the dynamic sequence of the measured needle shaft coordinate system (CS6) in the scanning plane coordinate system (CS3), namely, a dynamic sequence of the scanning plane to measured needle shaft transformation matrix (M3′). Optionally, the processing unit is further configured to perform an iterative computation based on minimizing losses between multiple dynamic sequences of the estimated needle shaft coordinate systems (CS6′) and the dynamic sequence of the measured needle shaft coordinate system (CS6), to determine the final transducer tracker to scanning plane transformation matrix (M2) and the final needle tracker to needle tip transformation matrix (M4), enabling three-dimensional navigation across different ultrasound transducers and needles.

The present invention also aims to provide an ultrasound needle navigation method of improving accuracy and efficiency in determining transformation relationships among an ultrasound transducer, a needle, and a scanning plane during puncture navigation. The method executed through an ultrasound needle navigation system, which includes a transducer tracker at the tail of an ultrasound transducer, a needle tracker at the tail of a needle, a tracking station, a needle image detection module, and a processing unit. The transducer tracker corresponds to a transducer tracker coordinate system, and the needle tracker corresponds to a needle tracker coordinate system. The ultrasound transducer is configured to provide a scanning plane. The method includes the steps of: providing, via the tracking field generated by the tracking field generator (e.g. a tracking field generator coordinate system (CS1)) of the tracking station, a dynamic sequence of the transducer tracker coordinate system (CS2) and a needle tracker coordinate system (CS5) to the processing unit; obtaining, through the needle image detection module, a dynamic sequence of a measured needle shaft coordinate system (CS6) of the needle in the scanning plane from ultrasound image sequences detected by the ultrasound transducer and a dynamic sequence of a scanning plane to the measured needle shaft transformation matrix (M3′); generating multiple estimated transducer tracker to scanning plane transformation matrices (M2′) and multiple estimated needle tracker to needle tip transformation matrices (M4′) based on the dynamic sequence of the scanning plane to measured needle shaft transformation matrix (M3′), the dynamic sequence of the transducer tracker coordinate system (CS2) and the dynamic sequence of the needle tracker coordinate system (CS5), through the processing unit; and generating multiple dynamic sequences of estimated needle shaft coordinate systems (CS6′), based on the multiple estimated transducer tracker to scanning plane transformation matrices (M2′) and the multiple estimated needle tracker to needle tip transformation matrices (M4'), and perform iterative computation (e.g. based on minimizing losses between the multiple dynamic sequences of estimated needle shaft coordinate systems (CS6′) and the dynamic sequence of the measured needle shaft coordinate systems (CS6)), to determine a final transducer tracker to scanning plane transformation matrix (M2) and a final needle tracker to needle tip transformation matrix (M4), enabling three-dimensional navigation across different ultrasound transducers and needles.

In summary, compared to the prior art, the present invention uses only two known transformation matrices (M1 and M5) together with an image-derived mapping M3′ (not one of the five closed-set core matrices) as inputs, and jointly—via an iterative procedure—determines the previously unknown M2 and M4. Specifically, the ultrasound needle navigation of the present invention, by executing the ultrasound needle navigation method, can collaborate with the user to calculate the transducer tracker to scanning plane transformation matrices, and needle tracker to needle tip transformation matrices, even without precise information of the needle tip coordinate system (only needing the needle shaft to be within the scanning plane), thereby addressing the problems of the prior art and adapting to various specifications of transducers and needles.

### BRIEF DESCRIPTION OF DRAWINGS

FIG. 1 is a system architecture diagram of an ultrasound needle navigation system according to an embodiment of the present invention;

FIG. 2 is a schematic diagram of multiple coordinate systems in the ultrasound needle navigation system according to an embodiment of the present invention;

FIG. 3 is a flowchart illustrating steps of the ultrasound needle navigation method executed by the ultrasound needle navigation system according to an embodiment of the present invention;

FIG. 4 is a detailed flowchart of step A3 in the ultrasound needle navigation method according to an embodiment of the present invention;

FIG. 5 is a detailed flowchart of step A5 in the ultrasound needle navigation method according to an embodiment of the present invention;

FIG. 6 is a conceptual diagram illustrating steps A7 to A9 according to an embodiment of the present application;

FIG. 7 is a schematic diagram illustrating the instruction-providing process of the system.

### SYMBOL DESCRIPTION

CS1 tracking field generator coordinate system
CS2 transducer tracker coordinate system
CS3 scanning plane coordinate system
CS4 needle tip coordinate system
CS5 needle tracker coordinate system
CS6 measured needle shaft coordinate system
CS3′ estimated scanning plane coordinate system
CS4′ estimated needle tip coordinate system
CS4'′ multilayer estimated needle tip coordinate system
CS6′ estimated needle shaft coordinate system
M1 tracking field generator to transducer tracker transformation matrix
M2 final transducer tracker to scanning plane transformation matrix
M3 scanning plane to needle tip transformation matrix
M4 final needle tracker to needle tip transformation matrix
M5 tracking field generator to needle tracker transformation matrix
M2′ estimated transducer tracker to scanning plane transformation matrix
M3′ scanning plane to measured needle shaft transformation matrix
M4′ estimated needle tracker to needle tip transformation matrix
1 ultrasound needle navigation system
2 tracking station
3 ultrasound main unit
10 tracking field generator
20 transducer tracker
30 ultrasound transducer
31 tail (of ultrasound transducer)
32 head (of ultrasound transducer)
40 needle tracker
50 needle
51 tail (of needle)
52 needle tip
53 needle shaft
60 scanning plane
70 needle image detection module
70CS2 transducer tracker physics-informed needle image detection module
80 processing unit
S20, S40 positioning signals
S60 ultrasound signal
3A, 70A, 80A-80E functions
TR1, TR1′-TR6′ tracking records
J_{D} positional dimension
K_{D} angular dimension
L_{D} time dimension
J1-J3 position intervals
K1-K2 angular intervals
L1-L4 time intervals

### DETAILED DESCRIPTION OF EMBODIMENT

Below is a detailed reference to the exemplary embodiment of the present invention, illustrated in the accompanying figures. Whenever possible, the same component symbols are used in the figures and descriptions to represent the same or similar parts.

Throughout this specification and the appended claims, certain terms are used to refer to specific components. Those skilled in the art should understand that different manufacturers of sensing devices may refer to the same components by different names. This document is not intended to differentiate components that perform the same function but are named differently. In the following specification and claims, words such as “comprising,” “including,” and “containing” are open-ended terms and should be interpreted to mean “including, but not limited to . . . ”.

The terms “about,” “substantially,” or “approximately” are generally interpreted as being within 10% of the given value or range, or within 5%, 3%, 2%, 1%, or 0.5% of the given value or range.

The use of ordinal numbers such as “first,” “second,” etc., in the specification and claims to modify elements is not intended to imply any order among these elements, nor does it imply an order between one element and another or in the manufacturing process. The use of these ordinals is only to make clear distinctions between elements with similar names. The same terms may not be used in the claims and the specification, and accordingly, the first component in the specification may be the second component in the claims.

Unless otherwise defined, all terms used herein (including technical and scientific terms) have the same meaning as commonly understood by those skilled in the art to which the present invention pertains. It should be understood that such terms, for example, as defined in commonly used dictionaries, should be interpreted consistently with the related technology and the context of the present invention, rather than in an idealized or overly formal sense, unless specifically defined in the embodiments of the present invention.

Furthermore, in the present invention, descriptions such as “when . . . ” or “while . . . ” indicate situations like “at the moment, before, or after,” and are not limited to occurrences happening simultaneously, as clarified herein. Descriptions like “disposed on . . . ” in the present invention indicate a positional relationship between two elements and do not restrict whether the two elements are in direct contact, unless explicitly specified, as clarified herein. Moreover, when multiple effects are described in the present invention and the word “or” is used between effects, it means that each effect may exist independently, but it does not preclude the simultaneous existence of multiple effects.

Additionally, terms such as “system,” “device,” “apparatus,” “module,” or “unit” used herein refer to an electronic component or a digital circuit composed of multiple electronic components, an analog circuit, or other broader circuit (e.g., an electronic product containing a circuit). Unless specifically indicated otherwise, these terms do not necessarily imply a hierarchical or level relationship. Furthermore, they can be implemented through suitable hardware and/or software and their appropriate configuration.

In addition, the names of various messages mentioned herein are only used for convenience in describing their functions and do not limit the nature of the message.

Besides, the term “unknown initially” or “uncertain” as used herein refers to matrices whose exact values are not predetermined with sufficient precision for direct use and therefore require iterative refinement or adjustment during system operation. Moreover, the term ‘estimated’ refers to a temporary computational result updated iteratively until convergence.

In one embodiment, a “dynamic sequence” of a coordinate system (e.g., CS6′) refers to a time series of poses recorded at discrete timestamps t₁, t₂, . . . , each representing an instantaneous pose of the coordinate system CS6′. Accordingly, a single CS6′ denotes the pose of the estimated needle shaft coordinate system at one specific time instant, while one dynamic sequence comprises multiple such instantaneous poses of CS6′ along the time axis. In one embodiment, the system may contain J×K×L×T coordinate instances, corresponding respectively to J positional indices, K angular indices, L temporal intervals, and T discrete timestamps within each dynamic sequence. In one embodiment, the processing unit can generate JKL kinds of dynamic sequences of estimated needle shaft coordinate systems CS6′, each representing a temporal collection of time-varying poses in the spatiotemporal domain.

For consistency with the Symbol Description, terms such as “coordinate system,” “transformation matrix,” and “pose” may be written in the singular form (e.g., “the coordinate system CS4”) even where multiple time-indexed instances are discussed. Thus, “a dynamic sequence of the coordinate system CS4” means the time-indexed set of instantaneous poses of CS4 over time. Unless expressly stated otherwise, the singular includes the plural and vice versa. Parenthetical labels (e.g., CS1-CS6, M1-M5, CS4′, CS4″) are cross-reference tags to the Symbol Description and by themselves carry no quantity. Where multiple instances are intended, this is indicated by accompanying temporal or enumeration language in prose (e.g., “at a given time point,” “for each time point,” “over J positions and K angles,” “across L intervals,” “over time”), without introducing new symbol variants or indices. When modifiers such as “final,” “single,” “unique,” “fixed,” or “time-invariant” are used, they indicate one instance and exclude the plural. For the avoidance of doubt, where the plural form is used expressly (e.g., “coordinate systems”), that plural usage is intentional and governs the interpretation of that phrase, indicating multiple instances of the named type. Nothing in this section narrows such expressly plural usages. When a plural noun is followed by a parenthetical label (e.g., “coordinate systems (CS6′)”), the label identifies the type defined in the Symbol Description and does not alter number; the plural denotes multiple instances of that type. This convention applies to both the description and the claims.

Direction, uniqueness, and terminology. Under the measurement model described in this specification, for any two parts—each having its own coordinate system—there exists a single, uniquely determined transformation relating them. Accordingly, whether an implementation computes the mapping from Part A to Part B or from Part B to Part A is immaterial for technical purposes; each uniquely determines the other. For readability only, this specification labels transformations in the form “from [source] to [target].” This is a naming convention and does not reflect any technical preference or limit the scope. Implementations that compute one orientation and then use the corresponding reverse mapping are equivalent and are intended to fall within the scope of this disclosure. To avoid ambiguity in long sentences, we consistently name transformations “from [source] to [target]” and avoid compressed phrases such as “X and Y transformation matrix.” Because each part has its own coordinate system, “from Part X to Part Y” means “from the coordinate system of Part X to the coordinate system of Part Y,” which may also be described as expressing the pose of the coordinate system of Part Y with respect to the coordinate system of Part X. A dynamic sequence of a coordinate system means a time series of such poses (i.e., ordered position-and-orientation records) with respect to the stated reference coordinate system.

Moreover, when multiple effects (or components) are described herein, if the word “or” is used between multiple effects (or components), it means that each effect (or component) may exist independently, but it does not preclude the possibility of multiple effects (or components) existing simultaneously.

It should be noted that, without departing from the spirit of the present invention, features from several different embodiments can be substituted, reorganized, or combined to form other embodiments. As long as the features do not contradict or violate the spirit of the invention, they can be freely mixed and matched across embodiments.

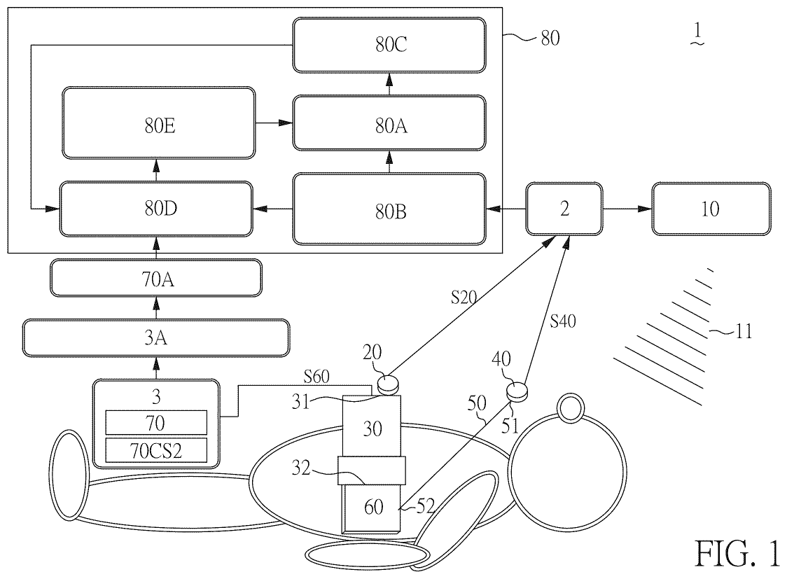

FIG. 1 is a schematic diagram of an ultrasound needle navigation system 1 (hereinafter referred to as the system 1) according to an embodiment of the present invention. As shown in FIG. 1, the system 1 may include a tracking station 2, a transducer tracker 20, a needle tracker 40, a needle image detection module 70, and a processing unit 80. The transducer tracker 20 can be disposed at the tail 31 of an ultrasound transducer 30, and the needle tracker 40 can be disposed at the tail 51 of a needle 50. A head 32 of the ultrasound transducer 30 can emit ultrasound sound waves, which propagate through a body, and returning ultrasound echo signals can form a scanning plane 60, which can be used for guiding the needle 50. It should be noted that, in this context, the term “guiding” is related to providing direction on a two-dimensional (2D) plane, for instance, offering guidance to position the needle 50 in a specified location, though it is not limited to this. In an embodiment, the term “navigation” refers to guidance within a three-dimensional (3D) space, though it is not limited to this. In an embodiment, the system 1 can also incorporate or be used with an ultrasound main unit 3, a tracking field generator 10, the ultrasound transducer 30, and/or the needle 50. The aforementioned components are merely examples, and can be reasonably added or removed. Moreover, in an embodiment, the system 1 may further include a transducer tracker physics-informed needle image detection module 70CS2, or the needle image detection module 70 and transducer tracker physics-informed needle image detection module 70CS2 may be integrated, thus expanding the types of signals, ultrasound scanning plane 60 and the coordinate system CS2, that can be processed for needle detection and is not intended to be limiting in any way.

Regarding the tracking station 2. In an embodiment, the tracking station 2 may be an electronic device equipped with communication equipment and/or a processor, such as a computer, a smart mobile device, or other electronic instruments, though it is not limited to this. In an embodiment, the tracking field generator 10 and/or the processing unit 80 may be integrated into the tracking station 2 but can also be disposed externally. In an embodiment, the tracking station 2 may also be configured or equipped with a camera module, and is not intended to be limited in this way.

About the tracking field generator 10. In an embodiment, the tracking field generator 10 can provide a tracking field 11. The transducer tracker 20 and the needle tracker 40 can generate positioning signals S20 and S40, respectively, based on the tracking field 11, and transmit the positioning signals to the tracking station 2. The tracking station 2 can determine the positions of the transducer tracker 20 and the needle tracker 40 in space according to the positioning signals S20 and S40, thereby determining the position of the tail 31 of the transducer 30 and the tail 51 of the needle 50. In one embodiment, the tracking field 11 provided by the tracking field generator 10 can be an electromagnetic field or an optical field, and the positioning signals S20 and S40 generated by the transducer tracker 20 and the needle tracker 40 can be reflected signals of electromagnetic or optical signals, and is not intended to be limiting in this way. In one embodiment, the tracking field 11 generated by the tracking field generator 10 of the tracking station 2 is an electromagnetic field, acting on the transducer tracker 20 to generate the positioning signal S20 and acting on the needle tracker 40 to generate the positioning signal S40. These signals can be interpreted by the tracking station 2 to obtain dynamic sequence of the coordinate system CS2 and dynamic sequence of the coordinate system CS5 in space. In another embodiment, the tracking field 11 generated by the tracking field generator 10 of the tracking station 2 is an optical field, acting on the transducer tracker 20 to generate an optical reflection positioning signal S20 and acting on the needle tracker 40 to generate an optical reflection positioning signal S40. These signals can, for example, be interpreted by a camera module configured by the tracking station 2 to obtain the dynamic sequence of the coordinate system CS2 and the dynamic sequence of the needle tracker coordinate system CS5 in space.

About the ultrasound transducer 30. In an embodiment, the head 32 of the ultrasound transducer 30 can emit an ultrasound signal S60, thereby providing the scanning plane 60. The needle tip 52 of the needle 50 can be inserted into a human body for clinical intervention or surgery. Due to tissue penetrating characteristics of the ultrasound signal S60, a scanning plane coordinate system CS3 can be located inside a human body. When at least one part of the needle 50 inside the human body is in the scanning plane 60, the ultrasound main unit 3 can execute a function 3A: obtaining a time-series of 2D ultrasound images of the scanning plane 60, so as to allow a medical personnel to observe the portion of the needle 50 on a screen connected to the ultrasound main unit 3.

About the needle image detection module 70 and the transducer tracker physics-informed needle image detection module 70CS2. In an embodiment, the needle image detection module 70 can detect the position of the needle 50 in the ultrasound image, for example, finding at least a shaft part of the needle 50 in the scanning plane 60. The transducer tracker physics-informed needle image detection module 70CS2 can use the dynamic sequence of the coordinate system CS2 as a physical auxiliary signal to find the position of the needle 50 in the scanning plane 60 or obtain the dynamic sequence of a scanning plane to measured needle shaft transformation matrix M3′, without limitation. In an embodiment, the needle image detection module 70 and/or the transducer tracker physics-informed needle image detection module 70CS2 can include an algorithm and can be stored in a non-transitory computer-readable medium (e.g., but not limited to, memory, hard disk, optical disk, portable hard drive, cloud storage, etc.) as a computer program product with one or more instructions. When a processor executes the one or more instructions, the processor can implement various functions of the needle image detection module 70 and/or the transducer tracker physics-informed needle image detection module 70CS2 described herein, without limitation. In an embodiment, the needle image detection module 70 and/or the transducer tracker physics-informed needle image detection module 70CS2 can be disposed in the tracking station 2, the processing unit 80, or the ultrasound main unit 3, without limitation. In an embodiment, the needle image detection module 70 can execute a function 70A: obtaining a dynamic tracking record of a needle shaft (e.g., a certain part of the needle 50 in the scanning plane 60) from the time-series of 2D ultrasound images of the scanning plane 60. The dynamic tracking record can be further converted into the scanning plane to measured needle shaft transformation matrix M3′, and it is not intended to be limited in this way.

Regarding the processing unit 80. In an embodiment, the processing unit 80 can perform operations for processing or computing. In one embodiment, the processing unit 80 can obtain a 3D dynamic tracking record of the transducer tracker 20 and a 3D dynamic tracking record of the needle tracker 40 and perform an analysis. The processing unit 80 can also obtain the 2D movement tracking record of the needle image that generated by the part of the needle 50 in the scanning plane 60 thickness, and convert it into the scanning plane to the measured needle shaft transformation matrix M3′, and analyze the movement tracking record. The processing unit 80 can have various functions, including but not limited to obtain the transformation matrices of multiple coordinate systems, execute transformations between multiple coordinate systems, execute iterative algorithms, and execute other types of algorithms. In an embodiment, the processing unit 80 can, for example, be implemented as hardware, software, or firmware. When the processing unit 80 is hardware, it can be, for example, a processor or a similar component. When the processing unit 80 is software or firmware, it can be presented as a computer program product containing one or more instructions stored on a non-transitory computer-readable medium (e.g., but not limited to, memory, hard disk, optical disk, portable hard drive, cloud storage, etc.). When the processor executes the one or more instructions, it can realize the various functions of the processing unit 80 described herein, and is not intended to be limited in this way. In an embodiment, the processing unit 80 can perform multiple functions, such as a function 80A: performing transformation between coordinate systems by using transformation matrices, or a function 80B: obtaining the dynamic tracking record of the transducer tracker 20 or needle tracker 40, or a function 80C: estimating various possible dynamic sequences of the ultrasound transducer scanning plane 60 or the needle tip 52 of the needle 50, or a function 80D: executing iterative algorithms or other algorithms, or a function 80E: estimating transformation matrices (the term “estimate” refers to, for example, a speculative value rather than an actual measurement), etc., without limitation.

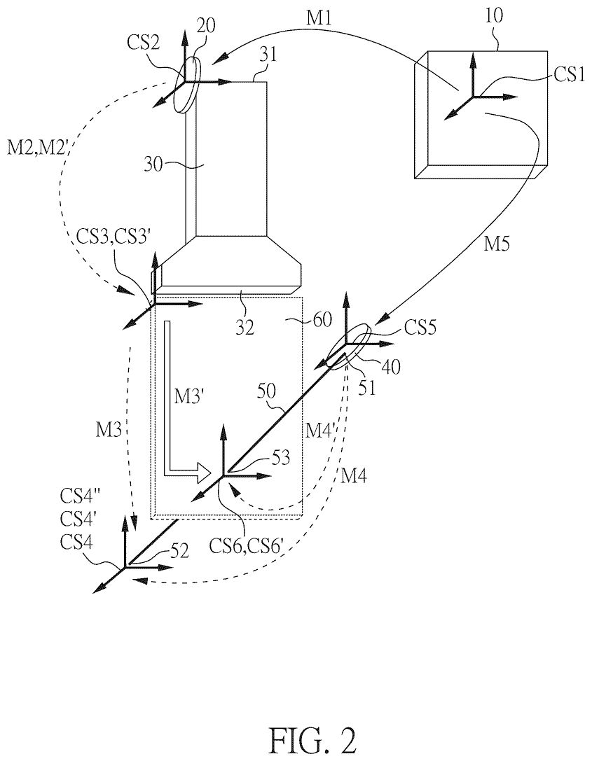

Next, the multiple coordinate systems in the system 1 are described. These coordinate systems can represent changes in the position and orientation of the transducer 30 and needle 50 in space. FIG. 2 is a schematic diagram of the multiple coordinate systems in the system 1 according to an embodiment of the present invention, and reference should also be made to FIG. 1.

As shown in FIG. 2, the tracking field generator 10 can correspond to a tracking field generator coordinate system CS1 (hereinafter referred to as the coordinate system CS1), the transducer tracker 20 can correspond to a transducer tracker coordinate system CS2 (hereinafter referred to as the coordinate system CS2), the scanning plane 60 can correspond to a scanning plane coordinate system CS3 (hereinafter referred to as the coordinate system CS3, which can be considered as the coordinate system of the head 32 of the transducer 30 or the emission origin of the ultrasound signal), the needle tip 52 of the needle 50 can correspond to a needle tip coordinate system CS4 hereinafter referred to as the coordinate system CS4, and the needle tracker 40 can correspond to a needle tracker coordinate system CS5 (hereinafter referred to as the coordinate system CS5, which can be considered as the coordinate system of the tail 51 of the needle 50).

The coordinate system CS1 and the coordinate system CS2 can have a tracking field generator to transducer tracker transformation matrix M1 (hereinafter referred to as the transformation matrix M1), to determine the relative pose of the transducer tracker 20 to the tracking field generator 10 in space. This transformation matrix M1 is derived from the tracking station 2 processing positioning signal S20 in the transducer tracker 20, which receives signals emitted from the tracking field generator 10. Thus, the tracking station 2 can acquire the pose of the transducer tracker 20 relative to the tracking field generator 10, and is not intended to be limited in this way. In one application scenario of the present invention, the coordinate system CS1 belongs to known static information, meaning that the pose of the tracking field generator 10 in space is fixed or static to the ground. The transformation matrix M1 belongs to a dynamic information known to the system, meaning that the pose of the transducer tracker 20 to the field generator is a known dynamic sequence of values.

Similarly, the coordinate system CS1 and the coordinate system CS5 can have a tracking field generator to needle tracker transformation matrix M5 (hereinafter referred to as the transformation matrix M5). This matrix M5 is to determine the relative pose of the needle tracker 40 to the tracking field generator 10 in space, and is derived from the tracking station 2 processing the positioning signal S40 from the needle tracker 40 which receives positioning signal from the tracking field generator 10. Therefore, the tracking station 2 can acquire the relative pose of the needle tracker 40 to the tracking field generator 10 in space, and is not intended to be limited in this way. In one application scenario of the present invention, the transformation matrix M5 is a known dynamic sequence of values.

A transducer tracker to scanning plane transformation matrix M2 (hereinafter referred to as the transformation matrix M2) may exist between the coordinate system CS2 and the coordinate system CS3. Using the transformation matrix M2, a dynamic sequence of the transducer tracker coordinate system CS2 can be converted to a dynamic sequence of the coordinate system CS3, and vice versa. Accordingly, when the processing unit 80 acquires the dynamic sequence of pose of the transducer tracker 20 (which can also be considered as the pose of the tail 31 of the transducer 30), it can use the transformation matrix M2 to obtain a specific dynamic sequence of pose of the scanning plane 60 (which can also be considered as the position information of the head 32 of the transducer 30), and vice versa. In one application scenario of the present invention, the transformation matrix M2 differs when the system 1 is used over various ultrasound's different transducers 30 with variable dimensions (transducer shapes and sizes). Therefore, during the initial application of the system 1 on a variable transducer 30 of various dimensions bonded with a variable attached transducer tracker 20, the transformation matrix M2 is an uncertain specific fixed value, for any transducer being used. Therefore, the dynamic sequence of the coordinate system CS3 can be derived from the dynamic sequence of coordinate system CS2 with an initially uncertain fixed transformation matrix M2.

The coordinate system CS3 and the coordinate system CS4 may have a scanning plane to needle tip transformation matrix M3 (hereinafter referred to as transformation matrix M3). Using the transformation matrix M3, the pose of the coordinate system CS3 can be converted to the pose of the coordinate system CS4, and vice versa. Therefore, when the processing unit 80 obtains specific pose information of the scanning plane 60 (which can also be considered as position information of the head 32 of the transducer 30), it can use the transformation matrix M3 to obtain the pose of the needle tip 52, and vice versa, and is not limited in this way.

In one embodiment, changes in the pose of the transducer 30 and the needle 50 in space can be represented using different formats, such as six degrees of freedom (DOF) or seven degrees of freedom, and is not limited thereto. The six degrees of freedom format is defined as: representing position using 3D coordinates (x, y, z) and representing orientation using three angles (roll angle, pitch angle, yaw angle). The seven degrees of freedom format is defined as: representing position using 3D coordinates (x, y, z) and representing orientation using four numbers (w, i, j, k), which can form a quaternion. In one embodiment, the transformation matrices M1 to M5 can support either six degrees of freedom or seven degrees of freedom, and is not limited thereto.

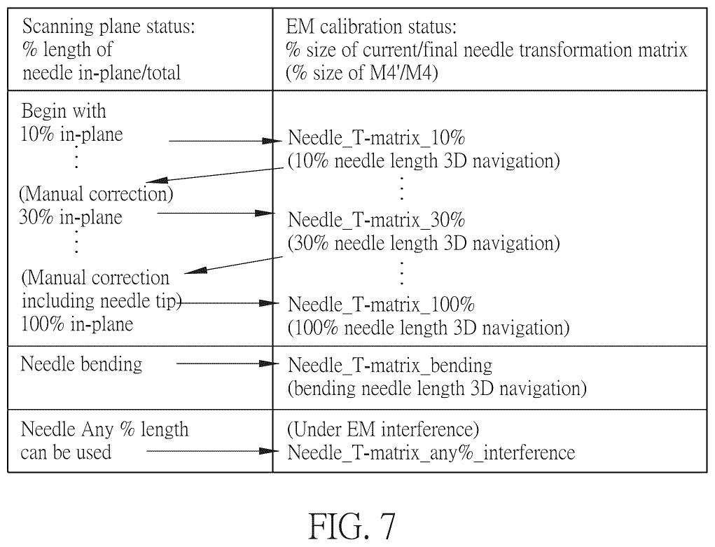

Furthermore, the pose of the needle 50 in space may change continuously during the surgery. Generally, an initial state called “partially in-plane” comes in as only the needle shaft 53 without its needle tip 52 lies in thickness of the scanning plane 60 in space. When the initially working system 1 hasn't solved the uncertain transformation matrix M2 and the uncertain needle tracker to needle tip transformation matrix M4 (discussed later), and the needle is only “partially in-plane” of its shaft, the needle image detection module 70 can only know a dynamic sequence of the scanning plane to measured needle shaft transformation matrix M3′, and a dynamic sequence of measured needle shaft coordinate system CS6. The desired coordinate system CS4 outside thickness of the scanning plane 60 and the scanning plane to needle tip transformation matrix M3 remains to be dynamic uncertain sequences, which through a process discussed later in FIG. 7, the needle tip 52 may completely lies in the thickness of the scanning plane 60, the needle image detection module 70 can detect a dynamic needle tip image in the scanning plane 60 and generate a dynamic sequence of transformation matrix M3 from the coordinate system CS3 to the coordinate system CS4, making both the coordinate system CS4 and the transformation matrix M3 a dynamic known information, as a feedback loop process discussed later in FIG. 7. In other words, the measured shaft transformation M3′ gradually converges to M3 once the tip appears in-plane.

The coordinate system CS4 and the coordinate system CS5 may have a needle tracker to needle tip transformation matrix M4 (hereinafter referred to as the transformation matrix M4). This transformation matrix M4 allows a dynamic pose of the coordinate system CS5 to be converted to a dynamic pose of the coordinate system CS4, and vice versa. Therefore, as long as the pose of the needle tracker 40 is known, the pose of the needle tip 52 can be transformed, and vice versa, and is not limited thereto. Additionally, when the needle image in the scanning plane 60 detected by the needle image detection module 70 is the needle shaft 53 rather than the needle tip 52, the measured needle shaft coordinate system CS6 (hereinafter referred to as the coordinate system CS6) and the coordinate system CS5 may have a needle tracker to measured needle shaft transformation matrix M4′, which is used as an iterative proxy for a needle tracker to an iteratively estimated needle tip transformation matrix. In one application scenario of the tip coordinate system CS4 remains to be uncertain dynamic sequence, the transformation matrix M4 remains to be an uncertain static transformation matrix, while the coordinate system CS6 and the needle tracker to measured needle shaft transformation matrix M4′ are known information and is updated iteratively in a feedback loop process discussed later in FIG. 7.

In FIG. 2, an arrow direction representing direction of transformation between two coordinate systems is only illustrative, and a solid arrow represents a known dynamic sequence of transformation matrix in the initial state of the system, while a dashed arrow indicates an uncertain transformation matrix to be solved. Three transformation matrices M2′, M3′, and M4′ represent estimated or measured transformation matrices used during the system's iterative search process, while another three transformation matrices M2, M3, and M4 represent the final transformation matrices established after the iterative search is complete.

Through the operation of the ultrasound needle navigation system 1 of the present invention, the processing unit 80 can calculate the uncertain transducer tracker to scanning plane transformation matrix M2, as well as the needle tracker to needle tip transformation matrix M4, with only the information of the transformation matrix M1, the scanning plane to measured needle shaft transformation matrix M3', and the transformation matrix M5. This demonstrates that, compared to the prior art, the present invention requires fewer known transformation matrices to derive more uncertain transformation matrices. Specifically, as described above, in the prior art, a 3D navigation system must obtain information of at least four transformation matrices to calculate the fifth transformation matrix. In contrast, the present invention uses two known transformation matrices (M1 and M5) together with an image-derived mapping M3′ (not one of the five closed-set core matrices) as inputs, and jointly—via an iterative procedure—determines the previously unknown M2 and M4.

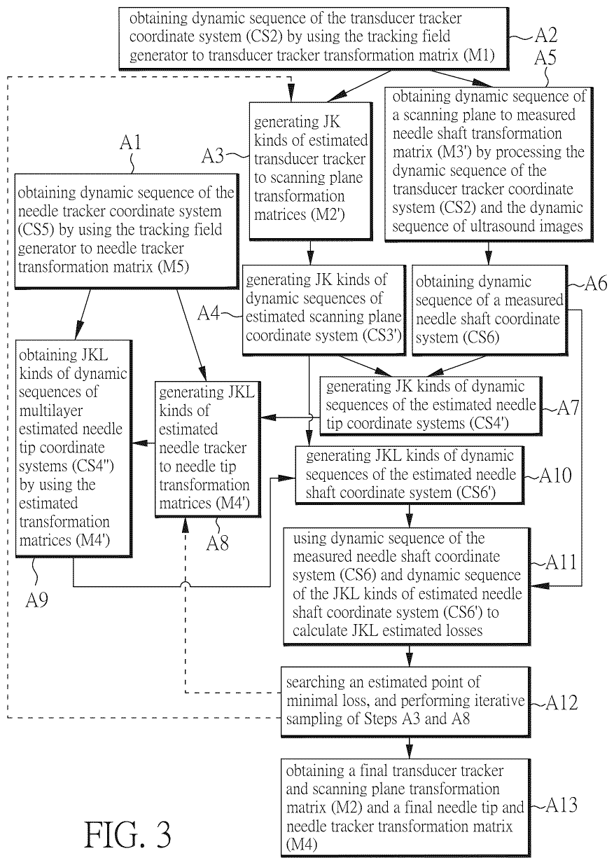

Next, the operation process of the ultrasound needle navigation system 1 will be described with FIG. 3, focusing on the method to estimate the final transducer tracker to scanning plane transformation matrix M2, as well as the needle tracker to needle tip transformation matrix M4. FIG. 3 is a schematic diagram of the operation process of the ultrasound needle navigation system 1 according to an embodiment of the present invention, and should be referred to alongside FIGS. 1 and 2.

As shown in FIG. 3, the ultrasound needle navigation method executed by the ultrasound needle navigation system 1 may include steps A1 to A13. Additionally, the processing unit 80 may operate in conjunction with a temporary storage unit (not shown in the figure), which can store various information generated during the steps. The temporary storage unit can be, for example, a memory or a hard drive, and can be located in the tracking station 2, ultrasound main unit 3, other electronic devices, or in the cloud, without limitation.

Regarding the step A1, the processing unit 80 uses the dynamic sequence records of the needle tracker 40 to obtain dynamic sequence of the transformation matrix M5, which may be considered as time-series pose of the needle tracker 40 in space (i.e., the pose of the needle tracker 40 relative to the tracking field generator 10). More specifically, the tracking station 2 can obtain the dynamic sequence records of the needle tracker 40 through the tracking field 11 generated by the tracking field generator 10. In one embodiment, the tracking station 2 (or processing unit 80) can obtain the pose signal S40 of the needle tracker 40 to derive the transformation matrix M5, which means deriving the dynamic sequence of the coordinate system CS5 from the positioning signal S40, without limitation.

Regarding the step A2, the processing unit 80 uses the dynamic sequence records of the transducer tracker 20 to obtain dynamic sequence of the transformation matrix M1, which can be considered as time-series pose of the transducer tracker 20 in space (i.e., the pose of the transducer tracker 20 relative to the tracking field generator 10). More specifically, the tracking station 2 can obtain the dynamic sequence records of the transducer tracker 20 through the tracking field 11 generated by the tracking field generator 10. In one embodiment, the tracking station 2 (or processing unit 80) can obtain the pose signal S20 of the transducer tracker 20 to derive the transformation matrix M1, which means deriving the dynamic sequence of the transducer tracker coordinate system CS2 from the positioning signal S20, without limitation.

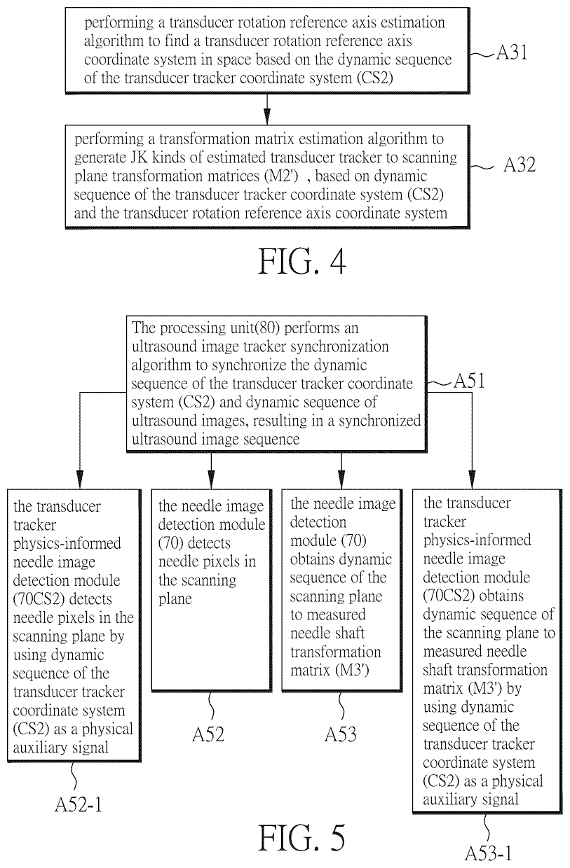

Regarding the step A3, after the step A2 is executed, the processing unit 80 generates JK kinds of estimated transducer tracker to scanning plane transformation matrices M2′. The step A3 may include multiple sub-steps, as explained in FIG. 4.

FIG. 4 is a detailed flowchart of the step A3 according to an embodiment of the present application, and should be referred to alongside FIGS. 1 to 3. As shown in FIG. 4, the step A31 is first executed, where the processing unit 80 performs a transducer rotation reference axis estimation algorithm to find a transducer rotation reference axis coordinate system CSRA in space based on the dynamic sequence of the transducer tracker coordinate system CS2. The details of the transducer rotation reference axis estimation algorithm will be described in subsequent paragraphs.

Next, the step A32 is executed, where the processing unit 80 performs a transformation matrix estimation algorithm to generate JK kinds of estimated transducer tracker to scanning plane transformation matrices M2′, based on the dynamic sequence of the coordinate system CS2 and the transducer rotation reference axis coordinate system CSRA (in this text, JK refers to J multiplied by K, wherein J and K are positive integers). When the transducer rotation reference axis coordinate system CSRA is established, J and K estimated points are sampled in two vector directions within the transducer rotation reference axis coordinate system CSRA to empirically cover the possible range of the transducer tracker to scanning plane transformation matrix, serving as a starting point for iterative minimization of a loss function. More specifically, when the user handles the transducer 30, it originally has six degrees of freedom in space. In this embodiment, J position points and K angles points are sampled along the transducer rotation reference axis coordinate system CSRA, equivalent to assuming that the transducer tracker is fixed at J positions and K angles on the transducer, resulting in J positions and K angles with respect to the scanning plane. Therefore, the original six degrees of freedom can be reduced to two degrees of freedom defined in the rotation-axis coordinate system CSRA,—J_{D} representing the positional degree of freedom along the rotation axis coordinate system CSRA, and K_{D} representing the angular degree of freedom about the rotation axis coordinate system CSRA, through the transducer rotation reference axis estimation algorithm and the transformation matrix estimation algorithm. J_{D} and K_{D} can be further mapped or projected onto the scanning plane 60, where J_{D} corresponds to a time-varying positional axis and K_{D} corresponds to a time-varying angular axis on the scanning plane 60, without limitation.

Regarding the step A4, please refer again to FIG. 3. After the step A3 is executed, the processing unit 80 generates JK kinds of estimated transducer tracker to scanning plane transformation matrices M2′ to convert one dynamic sequence of the transducer tracker coordinate system CS2 into JK kinds of dynamic sequences of the estimated scanning plane coordinate systems CS3'. Each of the JK kinds of dynamic sequences of the estimated scanning plane coordinate system CS3′ can be regarded as one estimated result of the processing unit 80, representing one of JK kinds of the time-series pose of the scanning plane 60 that is transformed by one of JK kinds of transformation matrices M2′ from one time-series pose of the ultrasound transducer 30 in space.

Regarding the step A5, the processing unit 80 (or tracking station 2) uses the ultrasound main unit 3 to obtain dynamic sequence of the scanning plane to measured needle shaft transformation matrix M3′. The step A5 may include multiple sub-steps, and may utilize dynamic sequence of the coordinate system CS2, which will be explained in conjunction with FIG. 5.

FIG. 5 is a detailed flowchart of the step A5 according to an embodiment of the present application, and should be referred to alongside FIGS. 1 to 3. As shown in FIG. 5, the step A51 is executed first, where the processing unit 80 performs an ultrasound image tracker synchronization algorithm to synchronize the dynamic sequence of the transducer tracker coordinate system CS2 with the dynamic sequence of ultrasound images from ultrasound transducer 30, resulting in a synchronized ultrasound image sequence. Then, the step A52 is executed, where the needle image detection module 70, installed in the ultrasound main unit 3, the processing unit 80, or the tracking station 2, detects needle image pixels (e.g., needle shaft 53) in the scanning plane 60 from the ultrasound image sequence. Subsequently, the step A53 is executed, where the needle image detection module 70 obtains a dynamic sequence of the scanning plane to the measured needle shaft transformation matrix (M3') from the ultrasound image sequence. The sequence of the steps A51, A52, and A53 is not fixed. Furthermore, the contents of the step A5 can be implemented using various existing technologies suitable for ultrasound needle navigation systems and are therefore not detailed here. Additionally, in one embodiment, the step A5 may also include the steps A52-1 and A53-1, where the step A52-1 involves the transducer tracker physics-informed needle image detection module 70CS2, installed in the ultrasound main unit 3, the processing unit 80, or the tracking station 2, using the dynamic sequence of the transducer tracker coordinate system CS2 as a physical auxiliary signal to detect needle image pixels (e.g., needle shaft 53) from the ultrasound image sequence. The step A53-1 involves the transducer tracker physics-informed needle image detection module 70CS2 using the dynamic sequence of the coordinate system CS2 as a physical auxiliary signal to obtain dynamic sequence of the scanning plane to measured needle shaft transformation matrix M3′, without limitation. In one embodiment, the step A52, A52-1, A53, A53-1 can be executed selectively, or all can be executed. The needle image pixels and the transformation matrix M3′ obtained with and without dynamic sequence of transducer tracker CS2 being used, can be cross-validated (various feasible cross-validation methods can be applied in this application) to improve accuracy, without limitation. Through the sub-steps of the step A5, the dynamic sequence of the scanning plane to measured needle shaft transformation matrix M3′ can be obtained.

Please refer again to FIGS. 3 and 5. Regarding the step A6, after executing the step A5, the processing unit 80 can obtain dynamic sequence of the coordinate system CS6 based on the dynamic sequence of the scanning plane to measured needle shaft transformation matrix M3′ and the needle image pixels identified(e.g., needle shaft 53) in the scanning plane 60. This is to present the dynamic pose of the needle shaft 53 within the scanning plane 60 relative to the scanning plane 60. In one embodiment, the step A6 can be considered as assigning spatial coordinate positions to the tracking record of the needle image pixel (e.g., needle shaft 53) detected in the scanning plane 60 in the step A5, without limitation.

Regarding the step A7, after executing the steps A4 and A6, the processing unit 80 projects the dynamic sequence of the coordinate system CS6 generated in Step A6 onto JK kinds of the 3D dynamic sequence of estimated scanning plane coordinate systems CS3′ generated in the step A4, to produce JK kinds of dynamic sequences of the estimated needle tip coordinate systems CS4′ in space. More specifically, for one ultrasound 2D frame sequence, this step uses JK kinds of transformation matrices to project the 2D needle shaft pixels of the coordinate system CS6 onto JK kinds of the 3D voxel frame sequences, consequently, to generate JK kinds of dynamic sequences of the estimated needle tip coordinate system CS4′ in space. In one embodiment, in each of the JK kinds of voxel needle in space, the voxel that is the farthest from the coordinate system CS5 or has certain specific characteristics can be considered as the estimated needle tip coordinate system CS4′ by the processing unit 80 during iteration, i.e., JK kinds of dynamic sequences of the estimated needle tip coordinate system CS4′ are generated.

Regarding the step A8, the processing unit 80 uses the dynamic sequence of the coordinate system CS5 generated in the step A1 and the JK kinds of dynamic sequences of the estimated needle tip coordinate system CS4′ generated in the step A7. Then, for each of the JK kinds of dynamic sequences of the estimated needle tip coordinate system CS4′, L kinds of estimated needle tracker to needle tip transformation matrices M4′ are provided, resulting in a total of JKL kinds of estimated needle tracker to needle tip transformation matrices M4′ (in one embodiment, the distal needle shaft is used as needle tip for building the transformation matrix M4′ from needle tracker). JKL refers to J multiplied by K multiplied by L, where J, K, and L are all positive integers. J is related to the spatial position dimension JD (e.g., J positions or position intervals), K is related to the spatial angle dimension KD (e.g., K angles or angle intervals), and L is related to the time dimension LD (e.g., L times or time intervals). In one embodiment, the dynamic sequence of the coordinate system CS5 represents the time-series pose of the needle tracker 40, and the dynamic sequence of the JK kinds of the estimated needle tip coordinate system CS4′ represents the time-series pose of the JK kinds estimated needle tips 52. In subsequent steps, through iterative computation and user collaboration, the transformation matrices M4′ are updated to iteratively decrease the losses of a loss function in the vector space JKL, to approximate the final needle tracker to needle tip transformation matrix M4. Additionally, in one embodiment, the range of values for the positive integers J, K, and L is determined by the performance of the computing device. The larger the value, the greater the number of transformation matrices tested, but the computational load also increases.

Regarding the step A9, after executing the step A8, the processing unit 80 uses the JKL kinds of estimated needle tracker to needle tip transformation matrices M4′ generated in the step A8 to convert the dynamic sequence of the coordinate system CS5 generated in the step A1 into JKL kinds of dynamic sequences of multilayer estimated needle tip coordinate systems CS4″. It should be distinguished from the estimated needle tip CS4′ generated in the step A7 by pixel to voxel projections (the pixels of the dynamic sequence of the coordinate system CS6 are projected over JK kinds of 3D dynamic sequence of scanning plane coordinate system CS3′ into JK kinds 3D voxels of the dynamic sequence of the estimated needle tip coordinate systems CS4′). The multilayer estimated needle tip coordinate systems CS4″ in the step A9 are directly converted from the dynamic sequence of the coordinate system CS5 by the JKL kinds of transformation matrices M4′.

As used herein, “multilayer” refers to a software-implemented multilayer estimation architecture that introduces image observations, geometric/tracker constraints, and temporal robustness at different layers, and does not require any physical material layers. In one embodiment, CS4 denotes the needle-tip coordinate system. CS4′ denotes a single-layer set of candidate estimated needle-tip coordinate systems generated, at a given time step, within a particular estimation layer and represented in a JK vector space that parameterizes transducer pose and related imaging features. CS4″ denotes a cross-layer set of candidate estimated needle-tip coordinate systems produced across one or more estimation layers and evaluated using a composite JKL loss that incorporates, in addition to JK terms, a temporal/robustness term L for consensus selection, temporal smoothing, and iterative feedback. In certain embodiments, the estimation layers iteratively update an estimated needle-tracker-to-needle-tip transformation M4′, and the composite JKL loss is used to select or reweight candidates within CS4′ and/or CS4″.

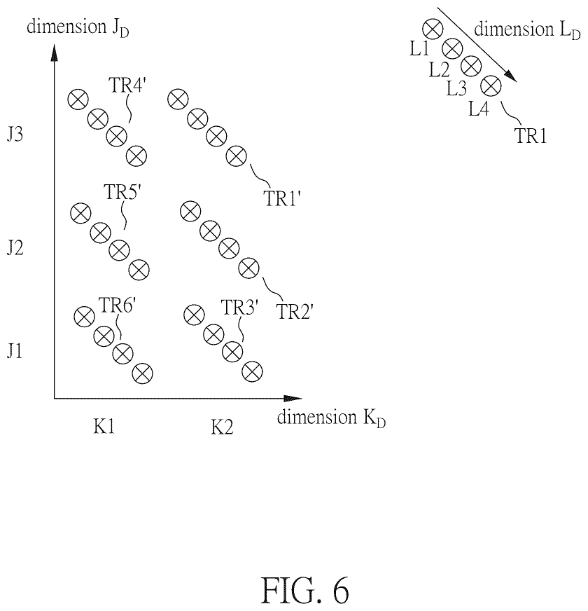

FIG. 6 is a detailed illustration of Steps A7˜A9 of an embodiment of the present application, and should be referred to alongside FIGS. 1 to 5. In FIG. 6, the tracking record TR1 of a measured needle tracker coordinate system CS5 of the needle tracker 40 in space is depicted. Additionally, the time dimension is represented by L_{D}, the positional dimension by J_{D}, and the angular dimension by K_{D}. The tracking record TR1 can include multiple subgroups according to the time dimension LD (e.g., four subgroups L1, L2, L3, L4, each corresponding to a time interval in the time dimension L_{D}), and each subgroup corresponds to one of the position intervals in the positional dimension JD (e.g., J1, J2, J3 each represent a position interval in the positional dimension J_{D}) and one of the angular intervals in the angular dimension K_{D} (e.g., two angular intervals K1, K2). This forms JK kinds of estimated needle tip coordinate systems CS4′ tracking record in space (e.g., J has three, K has two, resulting in six tracking record TR1′˜TR6′), and each tracking record in the JK kinds of tracking record CS4′ (e.g., TR1′, or when J=3 and K=2) can also be divided into L subgroups (e.g., four time intervals L1, L2, L3, L4 corresponding to TR1′). In this framework, each time subgroup of each tracking record in the JK tracking record (e.g., TR1′˜TR6′) (e.g., time interval L2 corresponding to TR1′) can provide L estimated needle tracker to needle tip transformation matrices M4′ for each of the JK estimated needle tip coordinate systems CS4′, generating a total of JKL estimated needle tracker to needle tip transformation matrices M4′ (as in Step A8). Subsequently, one dynamic sequence of the coordinate system CS5 can be converted into the JKL kinds of dynamic sequences of the multilayer estimated needle tip coordinate systems CS4″ (as in Step A9).

Please refer again to FIG. 3. Regarding the step A10, after executing the step A9, the processing unit 80 can use the JK kinds of dynamic sequences of estimated scanning plane coordinate system CS3′ generated in Step A4, and the JKL kinds of dynamic sequences of the multilayer estimated needle tip coordinate systems CS4'′ generated in the step A9, to produce JKL kinds of dynamic sequences of estimated needle shaft coordinate systems CS6′. In one embodiment, the processing unit 80 may, for example, draw both the scanning plane coordinate system CS3′ and the multilayer needle tip coordinate systems CS4″ (including the entire needle shaft extended by the vector of coordinate system CS4″) in space through 3D simulation, and turn the theoretical 3D crossover voxels into 2D pixels of the estimated needle shaft coordinate systems CS6′, thereby generating the JKL kinds of dynamic sequences of the estimated needle shaft coordinate systems CS6′, without limitation.

Regarding the step A11, after executing Step A10, the processing unit 80 can use one of the dynamic sequences of the coordinate system CS6 generated in the step A6, and the JKL kinds of dynamic sequences of estimated needle shaft coordinate systems CS6′ generated in the step A10, to calculate JKL kinds of estimated losses. In one embodiment, the processing unit 80 calculates JKL kinds of losses, between JKL kinds of estimated needle shaft coordinate systems CS6′ and the ground truth measured needle shaft coordinate system CS6.

Regarding the step A12, after executing the step A11, the processing unit 80 searches for a first generation of estimated point of minimal loss (Jₘ₁, Kₘ₁, Lₘ₁) in the JKL estimation loss vector space. The processing unit 80 performs iterative sampling of the steps A3 and A8 to find the second generation of estimated point of minimal loss (Jₘ₂, Kₘ₂, Lₘ₂), and so on, until the estimated loss is less than a set value or a loss threshold, or until the iteration count reaches a threshold value, ending the iteration process. The processing unit 80 can reach the latest estimated point of minimal loss (Jₘ, Kₘ, Lₘ) by gradient descent of loss after certain times of iteration. In one embodiment, the iteration process may be implemented through a type of neural network or an algorithm, without limitation. In one embodiment, the system 1 continues treating the needle shaft 53 as the needle tip 52 and provides guidance (e.g., messages provided on the ultrasound main unit 3, ultrasound transducer 20, tracking station 2, or other electronic devices, without limitation) to guide the user to insert more of the needle shaft 53 into the scanning plane, forming a positive feedback loop until the needle tip 52 is captured by the scanning plane. At this point, the final needle tip and needle tracker transformation matrix M4 is obtained, without limitation. In one embodiment, the processing unit 80 can display a 3D animation of the latest estimated point of minimal loss (Jₘ, Kₘ, Lₘ), in other words, displaying the best one of JKL kinds of multilayer estimated needle tip coordinate systems CS4″ and the best one of JK kinds of scanning plane coordinate systems CS3′, to assist the user placing the needle tip coordinate system CS4 into the scanning plane. In other words, the user adjusts relative pose between the needle 50 and the ultrasound transducer 30 to make estimated needle tip coordinate systems CS4′ and multilayer estimated needle tip coordinate systems CS4″ approach the needle tip coordinate system CS4.

Here is the process of the system 1 providing instructions to guide the user to insert more of the needle shaft 53 into the scanning plane. FIG. 7 is a schematic diagram of the process of the system 1 providing instructions to guide the user according to an embodiment of the present application, and please refer to FIGS. 1 to 6 at the same time. As shown in FIG. 7, in certain iteration of the algorithm, the system 1 may use latest iterated estimated transformation matrices M2′ and M4′ to provide the current 3D image to the user (for example, the image can be displayed on a screen). The user can relatively move the ultrasound transducer 30 and the needle 50 to increase the length of the needle (such as the needle shaft 53) shown in the scanning plane 60 thickness, thereby making the system 1 update (elongate) the estimated transformation matrix M4′ to be closer to the real transformation matrix M4, but is not limited thereto. In an embodiment, the system 1 can display 30% length of the needle 50 in the 3D image (e.g. the system 1 uses the transformation matrices M1 and M5, the estimated transformation matrix M2′, and 30% length version estimated transformation matrix M4′ to form the 3D and 2D display of the transformation matrix M3'), although the length of the displayed needle in the 3D image is shorter than the actual length, its trajectory is in the same direction as the actual needle 50 having the full length. Therefore, when the user aligns more the needle shaft 53 into the scanning plane thickness according to the shorter needle length version of the 3D image, it is equivalent to align more part of needle total length (for example, 40% to 100% of the length) into the scanning plane at the same time, thus needle adjustment time can be reduced.

Bending exception for transformation matrix M4. By default, for a rigid needle 50 and needle tracker 40 pairing within an operating/calibration interval, the transformation matrix M4 is a single time-invariant transformation; unless expressly stated otherwise, references to M4 elsewhere in this disclosure refer to this static transformation. When the needle bends (non-rigid deformation), the physical relationship between the needle tracker 40 and the needle tip 52 changes; accordingly, M4 is modeled as an instantaneous effective transformation that varies over time. The system updates M4 in real time to reflect bending so that the pose of the bent needle tip 52 can be obtained using the updated M4. When bending ceases or a new steady condition is established, the system may re-establish a static M4 for subsequent operation. For completeness, M4 denotes the mapping from the needle tracker 40 to the needle tip 52 regardless of whether bending occurs; in an embodiment, the present disclosure treats M4 as static in the rigid case and time-varying in the bending case. This bending exception statement applies only to M4 and, in the event of any inconsistency, this paragraph shall control over any general references to “static M4” elsewhere in this disclosure; it does not narrow the scope of the claims.

In addition, in an embodiment, when electromagnetic interference occurs in the system 1, the transformation matrices M1 and M5 may be inaccurate, and the impact of electromagnetic interference can be partially compensated by the system 1 updating the transformation matrices M2 and M4, and is not limited thereto.

Regarding the step A13, please refer to FIGS. 3 and 4. After executing the step A12, the processing unit 80 can use the estimated point of minimal loss (Jₘ, Kₘ, Lₘ) to determine a final transducer tracker to scanning plane transformation matrix M2 using (Jₘ, Kₘ), and to determine a final needle tracker to needle tip transformation matrix M4 using (Jₘ, Kₘ, Lₘ). The final transformation matrices M2 and M4 generated in this step are the final output of the ultrasound needle navigation method (the steps A1 to A13).

Thus, by executing the ultrasound needle navigation method, the ultrasound needle navigation system 1 of the present invention can accurately calculate the transducer tracker to scanning plane transformation matrix M2, as well as the needle tracker to needle tip transformation matrix M4, even when both M2 and M4 are uncertain. Furthermore, this is achieved without needing precise initial information about the coordinate system CS4 in the scanning plane 60. Instead, a partially in-plane needle with a needle shaft 53 in the scanning plane 60 may initiate the process by generating the coordinate system CS6. Through iterative algorithm and user collaboration (e.g., the system 1 provides guidance to help the user move the transducer 30 to capture more of the needle in the scanning plane until the needle tip enters the scanning plane, allowing the system to refine the needle tracker to needle tip transformation matrix M4), the system can realize 3D navigation of the needle tip 52 and the scanning plane 60 by using the needle tracker 40 and the transducer tracker 20. This design addresses issues present in prior art and is applicable to various specifications of transducers 30 and needles 50. The iterative computation continues until convergence of M2 and M4 is achieved.

Next, the details of the transducer rotation reference axis estimation algorithm are explained. Note that the transducer rotation reference axis is not a physical axis that could be illustrated to a particular component, but an imaginary axis estimated from the time series pose of the transducer during operation, with different operator dependent features. In other words, transducer motion is at least partly user and operation dependent and may be the subject for vector space dimension reduction learned by the neural network. In one embodiment, the transducer rotation reference axis estimation algorithm may be implemented by the processing unit 80 using a type of neural network to analyze the dynamic sequence of the coordinate system CS2, thereby identifying a transducer rotation reference axis and its corresponding coordinate system. In one embodiment, the training process for such a neural network is as follows. Dynamic sequences of the coordinate system CS2 are collected while various operators hold transducers of different sizes to guide the needle insertion. For each transducer of variable sizes, the transducer tracker to scanning plane transformation matrix M2 is a predetermined ground truth, which is also the final output of the JK iteration search (as J positions and K angles mentioned previously). The neural network, however, is not trained to directly predict transformation matrix M2. Instead, the ground truth for the neural network is defined as a transducer rotation reference axis, which is defined as a certain axis that generates a JK vector space requiring the lowest number of iterations to converge to the predetermined transformation matrix M2. Accordingly, the neural network should learn, from the dynamic sequence of coordinate system CS2, to predict an axis that yields a JK space providing the most efficient iteration convergence toward transformation matrix M2. Specifically, the neural network may be trained with a loss function configured to penalize predicted axes that, given fixed integer values of J and K, require a greater number of iterations to converge to the predetermined transformation matrix M2.

In one embodiment, since the two uncertain transformation matrices M2 and M4 in the system each have six degrees of freedom, the computational complexity of solving both matrices directly may grow exponentially with respect to their degrees of freedom, for example up to X⁶ multiplied by Y⁶ if the complexity for each degree of freedom in matrix M2 is X and for matrix M4 is Y. To improve the efficiency of solving the transformation matrices, a Physics-Informed Neural Network (PINN) technique may be employed. Specifically, after inputting the dynamic transducer tracker data, the PINN predicts a transducer rotation reference axis and its corresponding coordinate system as an intermediary feature. Once this axis is established, the remaining computational problem can be reduced to a JKL vector space that is tested by the iteration process, as described in steps A1 to A13. The system then iteratively searches for the minimum loss point (Jₘ, Kₘ, Lₘ) by gradient descent or another optimization method. In one embodiment, the physics-informed constraints may incorporate known kinematic or geometric relationships between the tracker and the scanning plane, thereby improving the reliability of the predicted axis. Under optimal conditions, the network can analyze user operation characteristics in real time to generate the rotation axis, effectively reducing the original X⁶Y⁶ search space to a JKL space, as previously described.

In another embodiment, the transducer rotation reference axis estimation algorithm may be implemented by the processing unit 80 executing specific steps to determine the transducer rotation reference axis and its corresponding coordinate system. Since the transducer rotation reference axis is not a physical axis but rather the result of dimension reduction of the motion pattern of the coordinate system CS2 when the operator holds the transducer to locate the needle, it represents an operational characteristic of the transducer. In this case, the present invention may utilize traditional signal analysis and data fitting methods to determine the transducer rotation reference axis. For example, by applying frequency-domain analysis such as Fast Fourier Transform (FFT) or time-domain analysis such as autocorrelation, periodic characteristics in the transducer tracker coordinate system CS2 motion record can be analyzed to calculate the position and features of the transducer rotation reference axis. Additionally, curve fitting techniques (such as the least squares method) may be used to further optimize the estimation of these characteristics, thereby determining the position and range of the axis. Whether using the aforementioned traditional algorithms or the previously described machine learning-based techniques (such as Physics-Informed Neural Networks, PINN), the core of the present invention is to determine the transducer rotation reference axis and to reduce the search space from X⁶Y⁶ to the vector space of JKL estimated losses while simultaneously solving the two uncertain static transformation matrices M2 and M4 in spatial navigation. Therefore, whether the transducer rotation reference axis is calculated through traditional algorithms or advanced machine learning techniques, such implementations shall be considered within the scope of the present invention.

Thus, the ultrasound needle navigation system 1 and the ultrasound needle navigation method of the present invention can be understood. The ultrasound needle navigation system 1 of the present invention only needs to obtain the transformation matrix M1, the transformation matrix M5, and the scanning plane to measured needle shaft transformation matrix M3′, in order to calculate the transducer tracker and scanning plane transformation matrix M2, as well as the needle tip and needle tracker transformation matrix M4, thereby solving the problems present in the prior art.

In one embodiment, the present invention relates to structural features, which can be determined to fall within the scope of the present invention based on the presence or absence of components, component configuration, mechanical observation, and/or operational methods of the disputed product. The present invention also relates to features of procedural steps, which can at least be determined to fall within the scope of the present invention based on the operational methods of the disputed product or the information obtained by reverse engineering the disputed product, without limitation.

The details or features among the embodiments of the present invention can be freely combined and used as long as they do not violate or conflict with the spirit of the invention.

The aforementioned specific embodiments should be construed as merely illustrative, and not limiting the rest of the present disclosure in any way.

1. An ultrasound needle navigation system, comprising:
a transducer tracker, installed at a tail of an ultrasound transducer, corresponding to a transducer tracker coordinate system, wherein the ultrasound transducer is configured to provide a scanning plane, corresponding to a scanning plane coordinate system;
a needle tracker, installed at a tail of a needle, corresponding to a needle tracker coordinate system, wherein the needle is configured to provide a tip, corresponding to a needle tip coordinate system;
a tracking station, configured to generate a tracking field through a tracking field generator, and provide a dynamic sequence of the transducer tracker coordinate system and a dynamic sequence of the needle tracker coordinate system to a processing unit;
a needle image detection module, configured to obtain a dynamic sequence of a measured needle shaft coordinate system of the needle in the scanning plane from ultrasound image sequence detected by the ultrasound transducer, and obtain a scanning plane to measured needle shaft transformation matrix; and
the processing unit, configured to generate multiple estimated transducer tracker to scanning plane transformation matrices and multiple estimated needle tracker to needle tip transformation matrices based on the dynamic sequence of the transducer tracker coordinate system, the dynamic sequence of the measured needle shaft coordinate system, and the dynamic sequence of the needle tracker coordinate system;
wherein, the processing unit is further configured to generate multiple dynamic sequences of estimated needle shaft coordinate systems based on the multiple estimated transducer tracker to scanning plane transformation matrices and the multiple estimated needle tracker to needle tip transformation matrices, and calculate estimation losses and perform iterative computation using the multiple dynamic sequences of the estimated needle shaft coordinate systems and the dynamic sequence of the measured needle shaft coordinate system to determine a final transducer tracker to scanning plane transformation matrix and a final needle tracker to needle tip transformation matrix.

2. The ultrasound needle navigation system of claim 1, wherein the processing unit generates a transducer rotation reference axis coordinate system based on the dynamic sequence of the transducer tracker coordinate system, and generates multiple estimated transducer tracker to scanning plane transformation matrices based on the transducer rotation reference axis coordinate system and the dynamic sequence of the transducer tracker coordinate system.

3. The ultrasound needle navigation system of claim 2, wherein the processing unit generates multiple dynamic sequences of estimated scanning plane coordinate systems based on the multiple estimated transducer tracker to scanning plane transformation matrices and the dynamic sequence of the transducer tracker coordinate system, and generates multiple dynamic sequences of estimated needle tip coordinate systems based on the multiple dynamic sequences of the estimated scanning plane coordinate systems and the dynamic sequence of the measured needle shaft coordinate system.

4. The ultrasound needle navigation system of claim 3, wherein the processing unit generates multiple estimated needle tracker to needle tip transformation matrices based on the dynamic sequence of the needle tracker coordinate system and the multiple dynamic sequences of the estimated needle tip coordinate systems.

5. The ultrasound needle navigation system of claim 4, wherein the processing unit generates multiple dynamic sequences of multilayer estimated needle tip coordinate systems based on the dynamic sequence of the needle tracker coordinate system and the multiple estimated needle tracker to needle tip transformation matrices, and generates the multiple dynamic sequences of estimated needle shaft coordinate systems based on the multiple dynamic sequences of the estimated scanning plane coordinate systems and the multiple dynamic sequences of the multilayer estimated needle tip coordinate systems.

6. The ultrasound needle navigation system of claim 5, wherein the processing unit is configured to perform iterative updates on one of the multiple estimated transducer tracker to scanning plane transformation matrices and one of the multiple estimated needle tracker to needle tip transformation matrices based on the estimation losses between the multiple dynamic sequences of the estimated needle shaft coordinate systems and the dynamic sequence of the measured needle shaft coordinate system, thereby reducing the estimation losses in each generation and ultimately obtaining the final transducer tracker to scanning plane transformation matrix and the final needle tracker to needle tip transformation matrix.

7. The ultrasound needle navigation system of claim 1, wherein the needle image detection module further comprises a transducer tracker physics-informed needle image detection module, which analyzes the ultrasound image sequence detected by the ultrasound transducer and the dynamic sequence of the transducer tracker coordinate system to obtain a dynamic sequence of a measured needle shaft coordinate system of the needle in the scanning plane, and a scanning plane to measured needle shaft transformation matrix.

8. The ultrasound needle navigation system of claim 1, wherein the tracking field generated by the tracking field generator of the tracking station is an electromagnetic field that acts on the transducer tracker to generate a positioning signal and on the needle tracker to generate a positioning signal, which are interpreted by the tracking station to determine the dynamic sequences of the transducer tracker coordinate system and the needle tracker coordinate system in space.

9. The ultrasound needle navigation system of claim 1, wherein the tracking field generated by the tracking field generator of the tracking station is an optical field, that acts on the transducer tracker to generate its optical reflection positioning signal, and that acts on the needle tracker to generate its optical reflection positioning signal, which are interpreted by the tracking station to determine the dynamic sequences of the transducer tracker coordinate system and the needle tracker coordinate system in space.

10. The ultrasound needle navigation system of claim 1, wherein the processing unit is configured to iteratively calculate and converge to (i) a final transducer tracker to scanning plane transformation matrix and (ii) a final needle tracker to needle tip transformation matrix, each of which is initially unknown or uncertain, that is, not predetermined with sufficient precision, and is iteratively updated until convergence, thereby enabling three-dimensional navigation of a puncture process across different specifications of the ultrasound transducer and the needle.

11. An ultrasound needle navigation method, executed through an ultrasound needle navigation system comprising a transducer tracker installed at the tail of an ultrasound transducer, a needle tracker installed at the tail of a needle, a tracking station, a needle image detection module, and a processing unit, wherein the transducer tracker corresponds to a transducer tracker coordinate system, the needle tracker corresponds to a needle tracker coordinate system, the ultrasound transducer is configured to provide a scanning plane corresponding to a scanning plane coordinate system, and the needle is configured to provide a tip corresponding to a needle tip coordinate system, the method comprising the steps of:
providing, through the tracking field generated by the tracking field generator of the tracking station, a dynamic sequence of the transducer tracker coordinate system and a dynamic sequence of the needle tracker coordinate system to the processing unit;
obtaining, by the needle image detection module, a dynamic sequence of a measured needle shaft coordinate system of the needle in the scanning plane from ultrasound image sequence detected by the ultrasound transducer, and obtaining a scanning plane to measured needle shaft transformation matrix;
generating, by the processing unit, multiple estimated transducer tracker to scanning plane transformation matrices and multiple estimated needle tracker to needle tip transformation matrices based on the dynamic sequence of the transducer tracker coordinate system, the dynamic sequence of the measured needle shaft coordinate system, and the dynamic sequence of the needle tracker coordinate system; and
generating, by the processing unit, multiple dynamic sequences of estimated needle shaft coordinate systems based on the multiple estimated transducer tracker to scanning plane transformation matrices and the multiple estimated needle tracker to needle tip transformation matrices, and using the multiple dynamic sequences of the estimated needle shaft coordinate systems and the dynamic sequence of the measured needle shaft coordinate system to calculate estimation losses, and performing iterative computation by the processing unit to obtain a final transducer tracker to scanning plane transformation matrix and a final needle tracker to needle tip transformation matrix.

12. The ultrasound needle navigation method of claim 11, wherein the processing unit generates a transducer rotation reference axis coordinate system based on the dynamic sequence of the transducer tracker coordinate system, and further generates the multiple estimated transducer tracker to scanning plane transformation matrices based on the transducer rotation reference axis coordinate system and the dynamic sequence of the transducer tracker coordinate system.

13. The ultrasound needle navigation method of claim 12, wherein the processing unit generates multiple dynamic sequences of estimated scanning plane coordinate systems based on the multiple estimated transducer tracker to scanning plane transformation matrices and the dynamic sequence of the transducer tracker coordinate system, and generates multiple dynamic sequences of estimated needle tip coordinate systems based on the multiple dynamic sequences of the estimated scanning plane coordinate systems and the dynamic sequence of the measured needle shaft coordinate system.

14. The ultrasound needle navigation method of claim 13, wherein the processing unit generates multiple estimated needle tracker to needle tip transformation matrices based on the dynamic sequence of the needle tracker coordinate system and the multiple dynamic sequences of the estimated needle tip coordinate systems.

15. The ultrasound needle navigation method of claim 14, wherein the processing unit generates multiple dynamic sequences of multilayer estimated needle tip coordinate systems based on the dynamic sequence of the needle tracker coordinate system and the multiple estimated needle tracker to needle tip transformation matrices, and generates the multiple dynamic sequences of the estimated needle shaft coordinate systems based on the multiple dynamic sequences of the estimated scanning plane coordinate systems and the multiple dynamic sequences of the multilayer estimated needle tip coordinate systems.

16. The ultrasound needle navigation method of claim 15, wherein the processing unit is configured to perform iterative updates on one of the multiple estimated transducer tracker to scanning plane transformation matrices and one of the multiple estimated needle tracker to needle tip transformation matrices based on the estimation losses between the multiple dynamic sequences of the estimated needle shaft coordinate systems and the dynamic sequence of the measured needle shaft coordinate system, thereby reducing the estimation losses in each generation and ultimately obtaining the final transducer tracker to scanning plane transformation matrix and the final needle tracker to needle tip transformation matrix.

17. The ultrasound needle navigation method of claim 11, wherein the needle image detection module is further configured as a transducer tracker physics-informed needle image detection module, which analyzes the ultrasound image sequence detected by the ultrasound transducer and the dynamic sequence of the transducer tracker coordinate system to obtain a dynamic sequence of a measured needle shaft coordinate system of the needle in the scanning plane, and a scanning plane to measured needle shaft transformation matrix.

18. The ultrasound needle navigation method of claim 11, wherein the tracking field generated by the tracking field generator of the tracking station is an electromagnetic field that acts on the transducer tracker to generate a positioning signal and on the needle tracker to generate a positioning signal, which are interpreted by the tracking station to determine the dynamic sequences of the transducer tracker coordinate system and the needle tracker coordinate system in space.

19. The ultrasound needle navigation method of claim 11, wherein the tracking field generated by the tracking field generator of the tracking station is an optical field, that acts on the transducer tracker to generate its optical reflection positioning signal, and that acts on the needle tracker to generate its optical reflection positioning signal, which are interpreted by the tracking station to determine the dynamic sequences of the transducer tracker coordinate system and the needle tracker coordinate system in space.

20. The ultrasound needle navigation method of claim 11, the method being performed by a processing unit and comprising iteratively calculating and converging to (i) a final transducer tracker to scanning plane transformation matrix and (ii) a final needle tracker to needle tip transformation matrix, each of which is initially unknown or uncertain, that is, not predetermined with sufficient precision, and is iteratively updated until convergence, thereby performing three-dimensional navigation of the puncture process across different specifications of the ultrasound transducer and the needle.
